(19) United States
(12) Patent Application Publication
(10) Pub. No.: US 2010/0042032 A1
(43) Pub. Date: Feb. 18, 2010

(54) TENDON STRETCHER

(76) Inventor: Rodney Louis Tomczak, Columbus, OH (US)

Correspondence Address: GEORGE R. REARDON, 3356 STATION COURT, LAWRENCVILLE, GA, 30044, US

(21) Appl. No.: 12/539,007

(22) Filed: Aug. 11, 2009

Related U.S. Application Data
(60) Provisional application No. 61/088,700, filed on Aug. 13, 2008.

(51) Int. Cl. A61F 5/00

(52) U.S. Cl. 602/28

(57) ABSTRACT

A tendon stretcher is disclosed. The tendon stretcher includes a compression stocking and at least one band coupled to a distal end of the compression stocking and extending upwardly to the proximal end of the compression stocking to provide a tension and stretch one or more of the Achilles, posterior tibial, and peroneal tendons. In one embodiment, an open toed compression stocking is disclosed having a compression stocking, a lateral band coupled to a distal end of the compression stocking on a lateral plantar surface and extending upwardly and crossed over and operatively coupled to a medial surface at the proximal end of the compression stocking, and a medial band coupled to a distal end of the compression stocking on a medial plantar surface and extending upwardly and crossed over and operatively coupled to a lateral surface at the proximal end of the compression stocking.

### CROSS-REFERENCE TO RELATED APPLICATION(S)

The present non-provisional patent application claims the benefit of priority of U.S. Provisional Patent Application No. 61/088,700, which is entitled “OPEN TOED NIGHT TIME DEVICE TO STRETCH THE ACHILLES, POSTERIOR TIBIA, AND OR PERONEAL TENDON(S)”, which was filed on Aug. 13, 2008, and which is incorporated in full by reference herein.

### FIELD OF THE INVENTION

The technology described herein relates generally to the fields of stretching devices and stockings. More specifically, this technology relates to a device and associated methods to stretch the Achilles, posterior tibial, and/or peroneal tendons to assist athletes of all ages and elders prevent strain of these various tendons.

### BACKGROUND OF THE INVENTION

Athletes of all ages and the elder patients strain the Achilles, posterior tibial and/or peroneal tendons. Primary therapy is to stretch the tendons and prevent passive contraction during disuse. A primary example of disuse is sleeping hours when the foot relaxes and the tendons contract, the opposite of stretching.

Related patents known in the background art include the following: U.S. Pat. No. 7,115,105, issued to Cropper on Oct. 3, 2006, discloses an ankle control system. U.S. Pat. No. 6,641,549, issued to Darcey on Nov. 4, 2003, discloses a custom-moldable support for patellar tendonitis. U.S. Pat. No. 6,311,334, issued to Reinhardt et al. on Nov. 6, 2001, discloses a compression hose for the treatment of leg conditions. U.S. Pat. No. 6,080,124, issued to Falk et al. on Jun. 27, 2000, discloses a patella strap method. U.S. Pat. No. 5,865,776, issued to Springs on Feb. 2, 1999, discloses a knee brace having differential flexibility posterior and anterior panels. U.S. Pat. No. 5,897,520, issued to Gerig on Apr. 27, 1999, discloses a unitary dorsal night splint. U.S. Pat. No. 5,898,948, issued to Kelly et al. on May 4, 1999, discloses a support/sport sock. U.S. Pat. No. 5,843,010, issued to Bodmer on Dec. 1, 1998, discloses a heel and ankle appliance. U.S. Pat. No. 5,620,413, issued to Olson on Apr. 15, 1997, discloses a combination ankle brace and wrap. U.S. Pat. No. 5,520,628, issued to Wehr on May 28, 1996, discloses an ankle encompassing pressure orthosis. U.S. Pat. No. 5,464,384, issued to Cromartie on Nov. 7, 1995, discloses an Achilles tendon support brace. U.S. Pat. No. 5,263,923, issued to Fujimoto on Nov. 23, 1993, discloses a wearing article for wearing in pressed relation to a human body surface. U.S. Pat. No. 5,135,473, issued to Epler et al. on Aug. 4, 1992, discloses an Achilles tendon wrap. U.S. Pat. No. 5,060,641, issued to Jones on Oct. 29, 1991, discloses an apparatus and method for the treatment of flexural deformities, contracted tendons, and angular limb deformities in foals. U.S. Pat. No. 4,444,389, issued to Wrucke on Apr. 24, 1984, discloses a tendon stretching device. U.S. Pat. No. 4,172,456, issued to Zens on Oct. 30, 1979 discloses an anti-embolism stocking. U.S. Pat. No. 5,399,155, issued to Strassburg et al. on Mar. 21, 1995, discloses a static ankle plantar-flexion prevention device.

The foregoing patent and other information reflect the state of the art of which the inventor is aware and are tendered with a view toward discharging the inventor's acknowledged duty of candor in disclosing information that may be pertinent to the patentability of the technology described herein. It is respectfully stipulated, however, that the foregoing patent and other information do not teach or render obvious, singly or when considered in combination, the inventor's claimed invention.

### BRIEF SUMMARY OF THE INVENTION

In various exemplary embodiments, the technology described herein provides a device and associated methods to stretch the Achilles, posterior tibial, and/or peroneal tendon(s) to assist athletes of all ages and elders prevent strain of these various tendons.

In one exemplary embodiment, the technology described herein provides a tendon stretcher. The tendon stretcher includes a compression stocking and at least one band coupled to a distal end of the compression stocking and extending upwardly to the proximal end of the compression stocking to provide a tension and stretch at least one tendon. The compression stocking is open toed to provide freedom of movement to the toes and to prevent dorsiflexion of the toes. The compression stocking is worn from just proximal to the toes around the ball of the foot to just below the knee.

The at least one band can include first and second bands coupled to a distal end of the compression stocking and extending upwardly to the proximal end of the compression stocking to provide a tension and stretch at least one tendon. The at least one band can include a lateral band coupled to a distal end of the compression stocking on a lateral plantar surface and extending upwardly and crossed over and operatively coupled to a medial surface at the proximal end of the compression stocking and a medial band coupled to a distal end of the compression stocking on a medial plantar surface and extending upwardly and crossed over and operatively coupled to a lateral surface at the proximal end of the compression stocking.

The tendon stretcher includes a lateral plantar anchor point at which the lateral band is anchored to the lateral plantar surface of the compression stocking. The tendon stretcher includes a medial plantar anchor point at which the medial band is anchored to the medial plantar surface of the compression stocking. The plantar anchor points can include one or more of hook and loop fasteners, rivets, and stitching.

The tendon stretcher includes a medial surface anchor point at which the lateral band is anchored to the medial surface at the proximal end of the compression stocking after crossover and a lateral surface anchor point at which the medial band is anchored to the lateral surface at the proximal end of the compression stocking after crossover. The medial surface anchor point and the lateral surface anchor point can include one or more of hook and loop fasteners, rivets.

In another exemplary embodiment, the technology described herein provides an open toed compression stocking apparatus to stretch selectively the Achilles, posterior tibial, and peroneal tendons. The stocking apparatus includes a compression stocking, wherein the compression stocking is worn from just proximal to the toes around the ball of the foot to just below the knee, and wherein the compression stocking is open toed to provide freedom of movement to the toes and to prevent dorsiflexion of the toes; a lateral band, the lateral band coupled to a distal end of the compression stocking on a lateral plantar surface and extending upwardly and crossed over and operatively coupled to a medial surface at the proximal end of the compression stocking; and a medial band, the medial band coupled to a distal end of the compression stocking on a medial plantar surface and extending upwardly and crossed over and operatively coupled to a lateral surface at the proximal end of the compression stocking.

The stocking apparatus also includes a lateral plantar anchor point at which the lateral band is anchored to the lateral plantar surface of the compression stocking; a medial plantar anchor point at which the medial band is anchored to the medial plantar surface of the compression stocking; a medial surface anchor point at which the lateral band is anchored to the medial surface at the proximal end of the compression stocking after crossover; and a lateral surface anchor point at which the medial band is anchored to the lateral surface at the proximal end of the compression stocking after crossover. The plantar anchor points and the surface anchor points include at least one from the group consisting of: hooks and loop fasteners, rivets, and stitching.

In yet another exemplary embodiment, the technology described herein provides a method for selectively stretching one or more of the Achilles, posterior tibial, and peroneal tendons. The method steps include: utilizing an open toed compression stocking apparatus having a compression stocking; a lateral band, the lateral band coupled to a distal end of the compression stocking on a lateral plantar surface and extending upwardly and crossed over and operatively coupled to a medial surface at the proximal end of the compression stocking; and a medial band, the medial band coupled to a distal end of the compression stocking on a medial plantar surface and extending upwardly and crossed over and operatively coupled to a lateral surface at the proximal end of the compression stocking; wherein the compression stocking is worn from just proximal to the toes around the ball of the foot to just below the knee; wherein the compression stocking is open toed to provide freedom of movement to the toes and to prevent dorsiflexion of the toes; applying the open toed compression stocking apparatus to a lower leg of the wearer; adjusting a tension in each of the lateral and medial bands to accommodate the stretching of one or more tendons; and securing the lateral and medial bands to maintain the tension and stretch the one or more tendons.

The method steps can also include: crossing the lateral and medial bands in front of the shin; securing the lateral band to the medial surface at the proximal end of the compression stocking; securing the medial band to the lateral surface at the proximal end of the compression stocking; and applying a generally equal tension in each of the lateral and medial bands to accommodate the stretching of the Achilles tendon, thereby maintaining the foot in a dorsiflexed position.

The method steps can further include: crossing the lateral and medial bands in front of the shin; securing the lateral band to the medial surface at the proximal end of the compression stocking; securing the medial band to the lateral surface at the proximal end of the compression stocking; and applying with a greater tension the medial band to the lateral surface at the proximal end of the compression stocking than is applied to the lateral band to accommodate the stretching primarily of the posterior tibial tendon.

The method steps can further include: crossing the lateral and medial bands in front of the shin; securing the lateral band to the medial surface at the proximal end of the compression stocking; securing the medial band to the lateral surface at the proximal end of the compression stocking; and applying with a greater tension the lateral band to the medial surface at the proximal end of the compression stocking than is applied to the medial band to accommodate the stretching primarily of the peroneal tendon.

The method steps can further include: increasing further the tension in the lateral band at the medial surface at the proximal end of the compression stocking to evert the foot, thereby stretching the posterior tibial tendon in addition the Achilles tendon.

The method steps can further include: increasing further the tension in the medial band at the lateral surface at the proximal end of the compression stocking to invert the foot, thereby stretching one or more peroneal tendons in addition the Achilles tendon.

There has thus been outlined, rather broadly, the more important features of the technology in order that the detailed description thereof that follows may be better understood, and in order that the present contribution to the art may be better appreciated. There are additional features of the technology that will be described hereinafter and which will form the subject matter of the claims appended hereto. In this respect, before explaining at least one embodiment of the technology in detail, it is to be understood that the invention is not limited in its application to the details of construction and to the arrangements of the components set forth in the following description or illustrated in the drawings. The technology described herein is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the technology described herein.

Further objects and advantages of the technology described herein will be apparent from the following detailed description of a presently preferred embodiment which is illustrated schematically in the accompanying drawings.

### BRIEF DESCRIPTION OF THE DRAWINGS

The technology described herein is illustrated with reference to the various drawings, in which like reference numbers denote like device components and/or method steps, respectively, and in which:

FIG. 1 is a front side perspective view of a tendon stretcher for stretching the Achilles, posterior tibial, and/or peroneal tendons, illustrating, in particular, an open-toed stocking, tension bands, and rivet and hook-and-loop fasteners, according to an embodiment of the technology described herein;

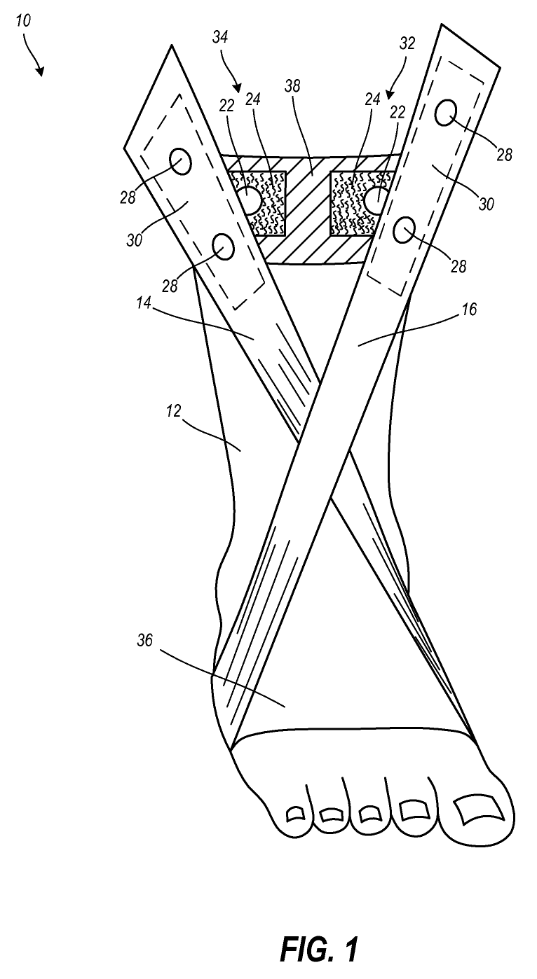

FIG. 2 is a left perspective view of the tendon stretcher depicted in FIG. 1, illustrating, in particular, the open-toed stocking, the tension bands, and rivet and hook-and-loop fasteners, according to an embodiment of the technology described herein; and

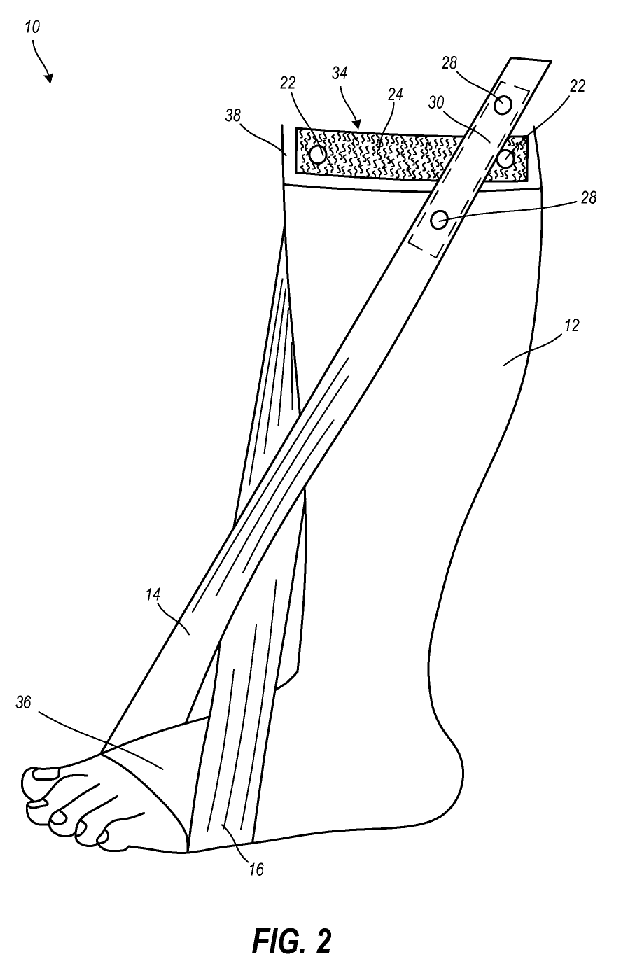

FIG. 3 is a bottom perspective view of the tendon stretcher depicted in FIG. 1, illustrating, in particular, the underside of the open-toed stocking, the tension bands, and rivet and hook-and-loop fasteners on the bottom of the stocking, according to an embodiment of the technology described herein.

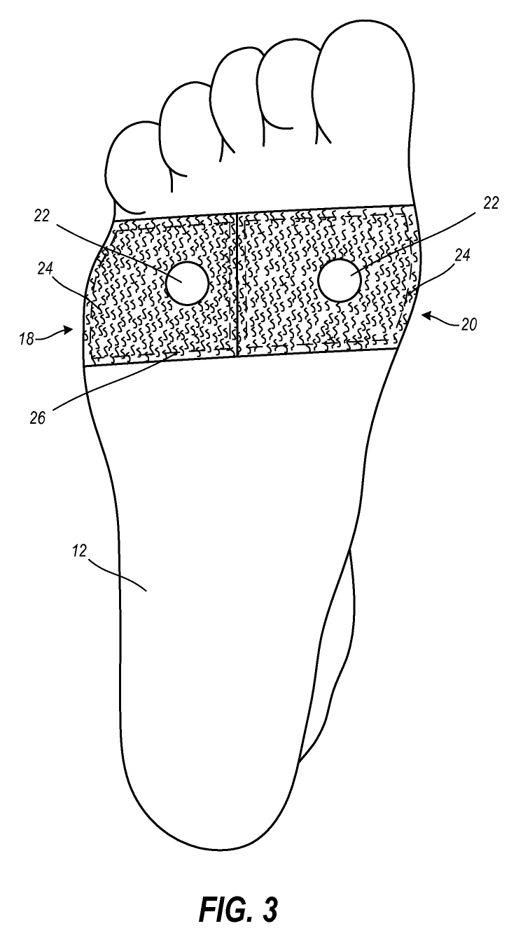

### DETAILED DESCRIPTION OF THE INVENTION

Before describing the disclosed embodiments of this technology in detail, it is to be understood that the technology is not limited in its application to the details of the particular arrangement shown here since the technology described is capable of other embodiments. Also, the terminology used herein is for the purpose of description and not of limitation.

In various exemplary embodiments, the technology described herein provides a device and associated methods to stretch the Achilles, posterior tibial, and/or peroneal tendon(s) to assist athletes of all ages and elders prevent strain of these various tendons. In an effort to prevent passive contraction during disuse of certain tendons, and tendonitis, it is desired to provide an apparatus and associated methods to stretch the Achilles, posterior tibial, and/or peroneal tendon(s).

Referring now to the Figures, a tendon stretcher 10 is shown. The tendon stretcher 10 includes a compression stocking 12. The compression stocking 12 preferably is open toed to provide freedom of movement to the toes and to prevent dorsiflexion of the toes. The compression stocking 12 is worn from just proximal to the toes around the ball of the foot to just below the knee. By way of example, the compression stocking 12 can be of mild to moderate compression such that it is enough compression to prevent the sliding down of the compression stocking 12 on the leg when the tension bands are connected. The compression stocking 12 can be constructed from nylon, latex, organic materials, or a combination thereof. The compression stocking 12 is manufactured from materials such that it can be laundered and cleaned.

The tendon stretcher 10 is designed to be flexible and comfortable to the wearer. Particularly as a device to be worn preferably at night while the wear is sleeping, the device avoids the use of the components of bulky, rigid, and cumbersome brace and cast type devices. The tendon stretcher 10 is configurable dynamically.

The tendon stretcher 10 includes at least one band coupled to a distal end 36 of the compression stocking 12 and extending upwardly to the proximal end 38 of the compression stocking 12 to provide a tension and stretch at least one tendon of the Achilles, posterior tibial, and/or peroneal tendon(s), the tendons that directly involve the ankle. The at least one band is manufactured from a durable, stretchable material.

The tendon stretcher 10 includes a lateral band 16. The lateral band 16 is coupled to a distal end 36 of the compression stocking 12 on a lateral plantar anchor point 18 and extends upwardly and crosses over to a medial surface 32 at the proximal end 38 of the compression stocking 12. The lateral band 12 can include one or more rivet orifice 28 to receive a rivet 22 and secure the lateral band 12. The lateral band 12 can include loop fasteners 30 to couple to the hook fasteners 24 on the proximal end 38 of compression stocking 12.

The tendon stretcher 10 includes a medial band 14. The medial band 16 is coupled to a distal end 36 of the compression stocking 12 on a medial plantar anchor point 20 and extends upwardly and crosses over to a lateral surface 34 at the proximal end 38 of the compression stocking 12. The medial band 16 can include one or more rivet orifice 28 to receive a rivet 22 and secure the medial band 16. The medial band 16 can include loop fasteners 30 to couple to the hook fasteners 24 on the proximal end 38 of compression stocking 12.

The size of the compression stocking 12 and the various lengths and widths of the lateral band 12 and the medial band 14 can be varied to accommodate varying sizes of the lower leg upon which the compression stocking 12 is worn.

The rivets 22 are secured to the compression stocking 12 such that they can securely hold the lateral band 16 and the medial band 14 in place. The rivets 22 can be manufactured from nickel, stainless steel, plastic, or the like, such that they are strong enough to securely hold the lateral band 16 and the medial band 14 in place.

The tendon stretcher 10 includes a lateral plantar anchor point 18 at which the lateral band 16 is anchored to the lateral plantar surface of the compression stocking 12. The tendon stretcher 10 includes a medial plantar anchor point 20 at which the medial band 14 is anchored to the medial plantar surface of the compression stocking 12. The plantar anchor points 18, 20 are located on the compression stocking 12 such that when the compression stocking 12 is worn, the plantar anchor points 18, 20 are located on the bottom of the foot at the metatarsal heads.

In various embodiments, the plantar anchor points 18, 20 further comprise hook fasteners 24 (to be coupled with loop fasteners 30), rivets 22, stitching 26, or the like. The placement of the hook fasteners 24 and the loop fasteners 30 can be reversed. Additionally, a combination of these fastening means can be utilized. The plantar anchor points 18, 20 can be formed from one strip of hook fasteners 24.

The tendon stretcher 10 includes a medial surface anchor point 32 at which the lateral band 16 is anchored to the medial surface at the proximal end 38 of the compression stocking 12 after crossover. The tendon stretcher 10 includes a lateral surface anchor point 34 at which the medial band 14 is anchored to the lateral surface at the proximal end 38 of the compression stocking 12 after crossover.

In various embodiments, the medial surface anchor point 32 and the lateral surface anchor point 34 further comprise further comprise hook fasteners 24 (to be coupled with loop fasteners 30), rivets 22, stitching 26, or the like. The placement of the hook fasteners 24 and the loop fasteners 30 can be reversed. Additionally, a combination of these fastening means can be utilized.

In operation an open toed compression stocking apparatus 10 can be utilized in various method steps to stretch selectively one or more of the Achilles, posterior tibial, and peroneal tendon(s). The lateral and medial bands 16, 14 are crossed in front of the shin. The lateral band 16 is secured to the medial surface anchor point 32 at the proximal end 38 of the compression stocking 12. The medial band 14 to the lateral surface anchor point 34 at the proximal end 38 of the compression stocking 12. A generally equal tension is applied in each of the lateral and medial bands 16, 14 to accommodate the stretching of the Achilles tendon, thereby maintaining the foot in a dorsiflexed position.

Alternatively, a greater tension can be applied to the medial band 14 at the lateral surface anchor point 34 at the proximal end 38 of the compression stocking 12 than is applied to the lateral band 16 in order to accommodate primarily the stretching of the posterior tibial tendon.

Alternatively, a greater tension can be applied to the lateral band 16 at the medial surface anchor point 32 at the proximal end 38 of the compression stocking 12 than is applied to the medial band 14 to accommodate primarily the stretching of the peroneal tendon.

In at least one method, the tension in the lateral band 16 at the medial surface anchor point 32 of the proximal end 38 of the compression stocking 12 is increased further in order to evert the foot, thereby stretching the posterior tibial tendon in addition the Achilles tendon.

In at least one method, the tension in the medial band 14 at the lateral surface anchor point 34 of the proximal end 38 of the compression stocking 12 is increased further in order to invert the foot, thereby stretching one or more peroneal tendons in addition the Achilles tendon.

By way of example, the open toed compression stocking apparatus 10 can be worn by the wearer while the wearer sleeps. Such usage allows for one or more tendons which directly involve the ankle to be stretched while the wearer sleeps and prevents passive contraction of the one or more tendons being stretched.

Although this technology has been illustrated and described herein with reference to preferred embodiments and specific examples thereof, it will be readily apparent to those of ordinary skill in the art that other embodiments and examples can perform similar functions and/or achieve like results. All such equivalent embodiments and examples are within the spirit and scope of the invention and are intended to be covered by the following claims.

What is claimed is:

1. A tendon stretcher comprising:
a compression stocking; and
at least one band coupled to a distal end of the compression stocking and extending upwardly to the proximal end of the compression stocking to provide a tension and stretch at least one tendon.

2. The tendon stretcher of claim 1, wherein the at least one band further comprises:
first and second bands, the first and second bands coupled to a distal end of the compression stocking and extending upwardly to the proximal end of the compression stocking to provide a tension and stretch at least one tendon.

3. The tendon stretcher of claim 1, wherein the at least one band further comprises:
a lateral band, the lateral band coupled to a distal end of the compression stocking on a lateral plantar surface and extending upwardly and crossed over and operatively coupled to a medial surface at the proximal end of the compression stocking; and
a medial band, the medial band coupled to a distal end of the compression stocking on a medial plantar surface and extending upwardly and crossed over and operatively coupled to a lateral surface at the proximal end of the compression stocking.

4. The tendon stretcher of claim 1, wherein the compression stocking is open toed to provide freedom of movement to the toes and to prevent dorsiflexion of the toes.

5. The tendon stretcher of claim 1, wherein the compression stocking is worn from just proximal to the toes around the ball of the foot to just below the knee.

6. The tendon stretcher of claim 3, further comprising:
a lateral plantar anchor point at which the lateral band is anchored to the lateral plantar surface of the compression stocking; and
a medial plantar anchor point at which the medial band is anchored to the medial plantar surface of the compression stocking.

7. The tendon stretcher of claim 6, wherein the plantar anchor points further comprise hook and loop fasteners.

8. The tendon stretcher of claim 6, wherein the plantar anchor points further comprise rivets.

9. The tendon stretcher of claim 1, further comprising:
a medial surface anchor point at which the lateral band is anchored to the medial surface at the proximal end of the compression stocking after crossover; and
a lateral surface anchor point at which the medial band is anchored to the lateral surface at the proximal end of the compression stocking after crossover.

10. The tendon stretcher of claim 9, wherein the medial surface anchor point and the lateral surface anchor point further comprise hook and loop fasteners.

11. The tendon stretcher of claim 9, wherein the medial surface anchor point and the lateral surface anchor point further comprise rivets.

12. An open toed compression stocking apparatus to stretch selectively the Achilles, posterior tibial, and peroneal tendons, the stocking apparatus comprising:
a compression stocking, wherein the compression stocking is worn from just proximal to the toes around the ball of the foot to just below the knee, and wherein the compression stocking is open toed to provide freedom of movement to the toes and to prevent dorsiflexion of the toes;
a lateral band, the lateral band coupled to a distal end of the compression stocking on a lateral plantar surface and extending upwardly and crossed over and operatively coupled to a medial surface at the proximal end of the compression stocking; and
a medial band, the medial band coupled to a distal end of the compression stocking on a medial plantar surface and extending upwardly and crossed over and operatively coupled to a lateral surface at the proximal end of the compression stocking.

13. The open toed compression stocking apparatus of claim 12, further comprising:
a lateral plantar anchor point at which the lateral band is anchored to the lateral plantar surface of the compression stocking;
a medial plantar anchor point at which the medial band is anchored to the medial plantar surface of the compression stocking;
a medial surface anchor point at which the lateral band is anchored to the medial surface at the proximal end of the compression stocking after crossover; and
a lateral surface anchor point at which the medial band is anchored to the lateral surface at the proximal end of the compression stocking after crossover.

14. The open toed compression stocking apparatus of claim 13, wherein the plantar anchor points and the surface anchor points comprise at least one from the group consisting of: hooks and loop fasteners, rivets, and stitching.

15. A method for selectively stretching one or more of the Achilles, posterior tibial, and peroneal tendons, the method comprising:
utilizing an open toed compression stocking apparatus comprising: a compression stocking; a lateral band, the lateral band coupled to a distal end of the compression stocking on a lateral plantar surface and extending upwardly and crossed over and operatively coupled to a medial surface at the proximal end of the compression stocking; and a medial band, the medial band coupled to a distal end of the compression stocking on a medial plantar surface and extending upwardly and crossed over and operatively coupled to a lateral surface at the proximal end of the compression stocking; wherein the compression stocking is worn from just proximal to the toes around the ball of the foot to just below the knee; wherein the compression stocking is open toed to provide freedom of movement to the toes and to prevent dorsiflexion of the toes;
applying the open toed compression stocking apparatus to a lower leg of the wearer;
adjusting a tension in each of the lateral and medial bands to accommodate the stretching of one or more tendons; and
securing the lateral and medial bands to maintain the tension and stretch the one or more tendons.

16. The method of claim 15, further comprising:
crossing the lateral and medial bands in front of the shin;
securing the lateral band to the medial surface at the proximal end of the compression stocking;
securing the medial band to the lateral surface at the proximal end of the compression stocking; and
applying a generally equal tension in each of the lateral and medial bands to accommodate the stretching of the Achilles tendon, thereby maintaining the foot in a dorsiflexed position.

17. The method of claim 15, further comprising:
crossing the lateral and medial bands in front of the shin;
securing the lateral band to the medial surface at the proximal end of the compression stocking;
securing the medial band to the lateral surface at the proximal end of the compression stocking; and
applying with a greater tension the medial band to the lateral surface at the proximal end of the compression stocking than is applied to the lateral band to accommodate the stretching primarily of the posterior tibial tendon.

18. The method of claim 15, further comprising:
crossing the lateral and medial bands in front of the shin;
securing the lateral band to the medial surface at the proximal end of the compression stocking;
securing the medial band to the lateral surface at the proximal end of the compression stocking; and
applying with a greater tension the lateral band to the medial surface at the proximal end of the compression stocking than is applied to the medial band to accommodate the stretching primarily of the peroneal tendon.

19. The method of claim 16, further comprising:
increasing further the tension in the lateral band at the medial surface at the proximal end of the compression stocking to evert the foot, thereby stretching the posterior tibial tendon in addition the Achilles tendon.

20. The method of claim 16, further comprising:
increasing further the tension in the medial band at the lateral surface at the proximal end of the compression stocking to invert the foot, thereby stretching one or more peroneal tendons in addition the Achilles tendon.
